(19) United States
(12) Patent Application Publication
(10) Pub. No.: US 2026/0345141 A1
(43) Pub. Date: Nov. 5, 2026

(54) KITS AND USE THEREOF IN SURGICAL PROCEDURES FOR ENDOSCOPIC HARVESTING OF VESSELS

(71) Applicant: Marizyme, Inc., Jupiter, FL (US)

(72) Inventor: Satish Chandran, Jupiter, FL (US)

(21) Appl. No.: 19/118,157

(22) PCT Filed: Oct. 4, 2023
(86) PCT No.: PCT/US2023/034467

§ 371 Date: Apr. 3, 2025

Related U.S. Application Data
(60) Provisional application No. 63/413,212, filed on Oct. 4, 2022.

(51) Int. Cl. A61B 17/00; A01N 1/126; A61B 1/313

(52) U.S. Cl. CPC A61B 17/00; A01N 1/126; A61B 1/3137; A61B 2017/00778

(57) ABSTRACT

Provided are kits comprising a combination of a surgical device for dissection and removal of blood vessels from a patient's body, and an endothelial damage inhibitor composition for preserving vessel function, and more particularly to a combination of an endoscopic vessel harvesting device and an endothelial damage inhibitor composition for preserving vessel function, where the composition is shelf stable and can be reconstituted at point of use, including during a coronary artery bypass graft (CABG) surgery or peripheral bypass surgery.

### CROSS REFERENCE TO RELATED APPLICATIONS

This application claims the benefit of U.S. Provisional Application No. 63/413,212, filed on Oct. 4, 2022, which is hereby incorporated by reference for all purposes as if fully set forth herein.

### FIELD OF THE INVENTION

The present invention is directed to kits comprising a combination of a surgical device for dissection and removal of blood vessels from a patient's body, and an endothelial damage inhibitor composition for preserving vessel function, and more particularly to a combination of an endoscopic vessel harvesting device and an endothelial damage inhibitor composition for preserving vessel function, where the composition is shelf stable and can be reconstituted at point of use, including during a coronary artery bypass graft (CABG) surgery, peripheral bypass surgery, and the like.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions, or is known to be relevant to an attempt to solve any problem with which this specification is concerned.

The teachings of all the references cited herein are incorporated in their entirety by reference.

Vascular conduits are used as grafts for a variety of bypass surgical procedures including, but not limited to, peripheral vascular bypass surgery and coronary artery bypass grafting (CABG) surgery. In these procedures the saphenous veins of the legs or radial arteries can be harvested for subsequent use. Harvesting of vascular conduits for use in surgical procedures is well known in the surgical field. Vessel harvesting involves liberating the vessel from surrounding tissue and transecting smaller side branches, tying or ligating the vessel at a proximal site and a distal site, and then transecting the vessel at both sites before it is removed from the body.

Exemplary endoscopic methods and devices for performing vessel harvesting are discussed in detail, e.g., in U.S. Pat. Nos. 5,895,353, 6,162,173, 6,406,425, 6,471,638, 7,510,562, 7,662,162, 7,695,470, 8,480,696, and 11,141,055. Commercial vessel harvesting systems currently are available from manufacturers such as Terumo Cardiovascular (Ann Arbor, MI) Getinge AB (Goteborg, Sweden), and Saphena Medical (West Bridgewater, MA). Specific examples include the VirtuoSaph® and VirtuoSaph® Plus Endoscopic Vein Harvesting Systems (Terumo Cardiovascular, Ann Arbor, MI) Vaso View® 7xB System (Getinge A B, Goteborg, Sweden), and Venapax (Saphena Medical, West Bridgewater, MA).

The saphenous vein is one of the most commonly-used vascular conduits for CABG procedures, usually used with the pedicle removed, and the procedure typically used for harvesting can result in vascular damage (Samano et al., Asian Cardiovasc. Thorac. Ann. 29 (5): 457-467, 2021). Graft failure can occur within 12 to 18 months after CABG surgery. While open vein harvesting with a long incision can reduce vessel damage, increased infection rate and longer recovery time after the open vein harvesting procedure has resulted in more common use of endoscopic harvesting instead, because of the smaller incision, reduced rate of infection, and much shorter recovery time.

In addition to possible trauma to the vessel during pedicle removal, endoscopic harvesting of vessels can result in collateral vessel damage. These additional sources of damage to the vessel can include thermal damage and physical damage, such as shear applied during the extraction procedure, which can negatively impact the endothelial layer of the vessel. In some methods, CO₂ insufflation is used to create a subcutaneous tunnel to facilitate vessel harvesting (Zingaro et al., Interact. Cardiovasc. Thorac. Surg. 15:661-664, 2012). The carbon dioxide used can react with water or other liquid to produce carbonic acid, which can result in localized pH changes at the endothelial layer of the vessel resulting in damage. These potential sources of trauma or damage can result in cumulative negative impacts on the harvested vessel, and can reduce its overall quality and long-term patency.

The effect of harvesting technique on clinical outcomes is still a topic of debate. Early studies showed that endoscopic vein harvesting had exhibited lower 1-year saphenous vein graft patency and higher 1-year revascularization rates (Zenati et al., J. Thorac. Cardiovasc. Surg. 141 (2): 338-34, 2011). While open vein harvesting was associated with superior patency at 5 years compared to the achieved using endoscopic harvesting, higher wound complications were associated with open vein harvesting (Mirza et al., J. Vasc. Surg. 67:1199-1206, 2018).

Endothelial dysfunction is the primary determinant in the interrelated pathogenesis leading to vascular conduit failure. Graft failure is preceded by graft thrombosis, intimal hyperplasia and accelerated graft atherosclerosis, all of which are predicated upon previous functional and/structural impairments of the vascular conduit endothelium. It has been recognized that the choice of storage solution for intra-operatively harvested saphenous vein segments has a significant impact on endothelial structure and function and therefore graft patency. The inability of most of these solutions to adequately preserve the vascular conduit has been demonstrated by a number of investigators (Thatte et al., Ann. Thorac. Surg. 75 (4): 1145-1152, 2003); and Hussaini et al., J. Cardiothorac. Surg. 6:82-90, 2011). A 2011 ex vivo study by Wilbring et al. (Eur. J. Cardiothorac. Surg. 40 (4): 811-815, 2011) demonstrated that vascular function was completely abolished after conduits harvested from patients were stored in a buffered saline solution, which is commonly used for vascular conduit storage. The data revealed that endothelial cell function was also significantly reduced, yet these dysfunctional conduits were successfully used as grafts for bypass surgery.

Previous attempts have been made to reduce the rate of graft failure by treating the harvested vessel. For example, a clinical trial to test the efficacy of edifoligide, an E2F transcription factor decoy, was performed to assess its ability in preventing graft failure. Vein grafts were treated ex vivo with either edifoligide or placebo (buffered normal saline). The results indicated that edifoligide was no more effective than placebo in preventing graft failure (Alexander, JAMA 294 (19): 2446-2454, 2005).

U.S. Pat. No. 7,981,596 discloses tissue and organ preservation solutions, generally called GALA™, and named after three primary components: glutathionc, ascorbic acid and L-arginine. These solutions have been found to be useful for preserving vascular conduits such as arteries and veins.

The effect of GALA™ preservation solution on human saphenous vein segments has also been evaluated in ex vivo studies. While saline solutions typically used in clinical settings led to a profound decline in endothelial cell viability of vascular conduits such as the saphenous vein, GALA™ maintained endothelial function and structural viability for at least up to 24 hours (Thatte et al., 2003, Hussaini et al., 2011, and Thatte, J. Cardiothorac. Surg. 6:82-90, 2011). The ex vivo data demonstrated that better preservation of vascular conduits could be afforded through the use of GALA™ as a vascular conduit preservation solution. However, the GALA™ solution has a limited shelf life due to instability of the GALA™ solution.

Thus, there is a need for compositions and methods that can be used to minimize or prevent graft failure for vessels harvested using endoscopic methods that have increased shelf life.

### SUMMARY

Provided are kits that include an endothelial damage inhibitor composition that has increased shelf life that can be used to minimize or prevent graft failure for vessels harvested using endoscopic methods. The kits include an endothelial damage inhibitor composition and an endoscopic vessel harvesting device. The endothelial damage inhibitor composition can include a first precursor aqueous solution comprising a balanced salt solution that has a pH of about 7.4 to about 8.0, and does not include reduced glutathione, ascorbic acid, or L-arginine; and a second precursor solution comprising water, reduced glutathione, ascorbic acid, a sugar, and L-arginine and having a pH of about 3.0 to about 4.0, where a mixture of the first precursor aqueous solution and the second precursor solution provides an endothelial damage inhibitor composition having a pH of about 7.3.

The endothelial damage inhibitor composition of the kits can include a powder formulation, comprising reduced glutathione, L-ascorbic acid, a sugar, L-arginine, a buffering agent, and a physiologically acceptable salt, where the powder formulation when reconstituted at a point of use in a saline or sterile water media forms a reconstituted solution having a pH of about 7. The sugar can be selected from the group consisting of glucose, fructose, mannose and ribose. The physiologically acceptable salt can include calcium chloride, potassium phosphate monobasic, magnesium sulfate, magnesium chloride, sodium phosphate dibasic, potassium chloride; and sodium bicarbonate. The salts can be their anhydrous form. In the powder formulation of the endothelial damage inhibitor composition of the kits, the reduced glutathione, L-ascorbic acid, the sugar and the L-arginine can be included in the powder formulation in amounts that produce a reconstituted solution having a concentration of the L-arginine from about 250 μM to about 2000 μM, a concentration of the sugar from about 5 mM to about 50 mM, a concentration of the L-glutathione from about 50 μM to about 3000 μM, and a concentration of the L-ascorbic acid from about 500 μM to about 4000 μM. In some formulations, the powder formulation does not include sodium chloride.

Also provided are kits that include an endothelial damage inhibitor composition and an endoscopic vessel harvesting device, where the endothelial damage inhibitor composition includes a powder formulation, which includes a reducing agent, an antioxidant, a buffering agent, a physiologically acceptable salt, optionally, a sugar; and optionally, a nitric oxide substrate, where the powder formulation when reconstituted at a point of use in a saline or sterile water media forms a reconstituted solution having a pH of about 7. The reducing agent can be at least one selected from among a reduced glutathione and cysteinylglycine. The antioxidant can be ascorbic acid. When present, the sugar can be selected from the group consisting of glucose, fructose, mannose and ribose. When present, the nitric oxide substrate can be L-arginine.

Also provided are kits that include an endothelial damage inhibitor composition and an endoscopic vessel harvesting device, where the endothelial damage inhibitor composition includes a powder formulation, which includes reduced glutathione, L-ascorbic acid, D-glucose, L-arginine, a buffering agent, and a physiologically acceptable salt. The physiologically acceptable salt can include one or more of anhydrous calcium chloride, potassium phosphate monobasic, anhydrous magnesium sulfate, anhydrous magnesium chloride, anhydrous sodium phosphate dibasic, anhydrous potassium chloride, and sodium bicarbonate. The powder formulation can include reduced glutathione, L-ascorbic acid, the sugar and the L-arginine in amounts that produce the reconstituted solution having a concentration of the L-arginine from about 250 μM to about 2000 μM, a concentration of the sugar from about 5 mM to about 50 mM, a concentration of the L-glutathione from about 50 μM to about 3000 μM, and a concentration of the L-ascorbic acid from about 500 μM to about 4000 μM. The powder formulation can be formulated so that it does not include sodium chloride. In the kits provided herein where the endothelial damage inhibitor composition is provided in the form of a powder formulation, the powder formulation can be provided in a form that has been sterilized by gamma irradiation.

In the kits provided herein, any endoscopic vessel harvesting device known in the art can be included. The endoscopic vessel harvesting device can include a closed tunnel system that includes a balloon to occlude an access site and tubing to provide carbon dioxide to insufflate a dissection tunnel. The endoscopic vessel harvesting device can be an open system that does not occlude an access site or provide carbon dioxide to insufflate a dissection tunnel. The endoscopic vessel harvesting device can include a vessel dissector, a vessel harvester, a trocar, or a combination thereof. The endoscopic vessel harvesting device can include a conical vessel dissector tip, a spoon-shaped vessel dissector tip, or a bullet-shaped vessel dissector tip. The vessel dissector tip can be movable and can be transparent to permit viewing forward of the tip using an endoscope. The vessel harvester of the endoscopic vessel harvesting device can include one or more tools that are located within the vessel harvester and that permit manipulation, cutting, and sealing of vessels.

The kits provided herein can include instructions for use.

Also provided are methods of improving the quality of an endoscopically harvested vessel. The methods include reconstituting the endothelial damage inhibitor composition of the kits described herein in saline or a sterile water media to form a reconstituted solution, and soaking the endoscopically harvested vessel in the reconstituted solution for at least 15 minutes. The methods also can include flushing the endoscopically harvested vessel with the reconstituted solution after harvesting and prior to soaking. The methods also can include flushing the endoscopically harvested vessel with the reconstituted solution after soaking.

Also provided are methods of reducing damage of an endoscopically harvested vessel, the methods including reconstituting the endothelial damage inhibitor composition of the kits described herein in saline or a sterile water media to form a reconstituted solution, and administering the reconstituted solution via the endoscopic vessel harvesting device as a replacement for CO₂ insufflation, or in conjunction with CO₂ insufflation, to create a subcutaneous tunnel to facilitate vessel harvesting.

It should be understood that the various individual aspects and features of the present invention described herein can be combined with any one or more individual aspect or feature, in any number, to form embodiments of the present invention that are specifically contemplated and encompassed by the present invention.

### BRIEF DESCRIPTION OF THE DRAWINGS

FIG. 1 is a chart of patient groupings of Example 1.

FIG. 2 is a histogram of propensity scores demonstrating good matching between groups.

FIG. 3A is a graph of Kaplan Meier curves of MACE rates at 1-year.

FIG. 3B is a graph of Kaplan Meier curves of all death rates at 1-year.

FIG. 3C is a graph of Kaplan Meier curves of Myocardial infarction (MI) rates at 1-year.

FIG. 3D is a graph of Kaplan Meier curves of revascularization rates at 1-year.

FIG. 4A is a graph of wall thickness of endoscopic harvesting of SVH using DuraGraft® vascular graft treatment composition endothelial damage inhibitor versus saline.

FIG. 4B is a graph of wall thickness of open harvesting of SVH using DuraGraft® vascular graft treatment composition endothelial damage inhibitor versus saline.

FIG. 5A is a graph of maximal stenosis of endoscopic harvesting of SVH using DuraGraft® vascular graft treatment composition endothelial damage inhibitor versus saline.

FIG. 5B is a graph of maximal stenosis of open harvesting of SVH using DuraGraft® vascular graft treatment composition endothelial damage inhibitor versus saline.

FIG. 6A is a graph of vessel diameter of endoscopic harvesting of SVH using DuraGraft® vascular graft treatment composition endothelial damage inhibitor versus saline.

FIG. 6B is a graph of vessel diameter of open harvesting of SVH using DuraGraft® vascular graft treatment composition endothelial damage inhibitor versus saline.

### DETAILED DESCRIPTION

Further aspects, features and advantages of this invention will become apparent from the detailed description which follows.

### DEFINITIONS

As used herein, the singular forms “a”, “an” and “the” are intended to include the plural forms as well, unless the context clearly indicates otherwise.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics, up to and including a variation of ±10%. All of the values characterized by the above-described modifier “about,” are also intended to include the exact numerical values disclosed herein. Moreover, all ranges include the upper and lower limits.

Any compositions described herein are intended to encompass compositions which consist of, consist essentially of, as well as comprise, the various constituents identified herein, unless explicitly indicated to the contrary.

As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the end-points of the range. As an example, a variable which is described as having values between 0 and 10, can be 0, 4, 2-6, 2.75, 3.19-4.47, etc.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used herein, the terms “patient” and “subject” include members of the animal kingdom including, but not limited to, human beings.

As used herein, “sterile water” includes, but is not limited to, (a) sterile water for injection, USP, (b) sterile distilled deionized water, and (c) sterile water for irrigation.

As used herein, an “antioxidant” is a substance that, when present in a mixture or structure containing an oxidizable substrate biological molecule, delays or prevents oxidation of the substrate biological molecule. For example, ascorbic acid is an antioxidant.

As used herein, a “balanced salt solution” is defined as an aqueous solution that is osmotically balanced to prevent acute cell or tissue damage.

As used herein, a “buffered salt solution” is defined as a balanced salt solution to which chemicals have been added to maintain a predetermined physiological pH range.

As used herein, “GALA” or “GALA™ refers to a type of tissue preservation solution including glutathione, ascorbic acid, L-arginine and a balanced salt solution.

As used herein, “graft” is defined as tissue that is transplanted or implanted in a part of the body to repair a defect or to improve the appearance. A graft can be homologous (transplanted into the same patient from which it was originally removed) or heterologous (transplanted into a recipient different from the donor).

As used herein, a “harvested bypass conduit” is defined as a surgically installed alternate route for the blood to bypass an obstruction.

As used herein, a “buffering agent” is a chemical compound or compounds (generally a weak acid or base or combination thereof) that maintains the pH of a solution in a desired range. Examples of physiologically tolerated buffers are, for example, acetate, bicarbonate, citrate, sodium phosphate, succinate, histidine, sulfate, nitrate, and pyruvate buffers, but are not limited thereto.

As used herein, a “pH adjusting agent” is chemical compound that can raise or lower pH when added to a solution. Preferred pH adjusting agents include HCl for lowering pH and NaHCO₃ for raising pH, but are not limited thereto.

As used herein, an “isotonic solution” refers to a solution that has the same salt concentration as cells.

As used herein a “physiologic solution” is defined as an aqueous salt solution which is compatible with normal tissue, by virtue of being isotonic with normal interstitial fluids and having physiological pH.

As used herein, a “powder formulation” is defined as a dry, solid substance, composed of finely divided components (salts/organics) with or without excipients and intended for internal or external use. It is a solid substance and be provided in a lyophilized or in finely divided state typically obtained by crushing, grinding, milling and/or comminuting. In a preferred embodiment, the components of the powder formulation can be anhydrous.

As used herein, a “saline solution” is defined as an NaCl solution used in surgical and medical treatment processes.

### Endothelial Damage Inhibition Composition

The methods and kits provided herein include as a component an endothelial damage inhibition composition. As described in U.S. Pat. No. 11,291,201, it has been shown that stability of the GALA™ solutions can be improved by separating the formulation into a first Solution A including a balanced salt solution and having a pH of at least 7, generally 7.4-8, and a Solution B having a pH of less than 7, preferably about 5, 4, or 3. Solution B includes water, an antioxidant such as ascorbic acid, a reducing agent such as L-glutathione, a nitric oxide substrate such as L-arginine and a sugar such as D-glucose. Solution B preferably has an oxygen content at zero parts per million. In other stable embodiments, the sugar can be added to Solution A instead of Solution B. The Sterility Assurance Level (SAL) of aseptically processed products is 103. This stable two-solution formulation, commercially available as DuraGraft® vascular graft treatment composition, is currently manufactured as a two-solution-containing kit with an aseptically manufactured Solution A bottle and Solution B vial. Solutions A and B are mixed at the point-of-use (POU) to generate an endothelial damage inhibition composition. The containers of solutions A and B of the DuraGraft® vascular graft treatment composition can be easily packaged with an endoscopic vessel harvesting device to form the kits provided herein. DuraGraft® vascular graft treatment composition has been shown in clinical trials to have a favorable effect on harvested vessel wall thickness when the endoscopically harvested vessel was flushed with and stored in DuraGraft® vascular graft treatment composition for a minimum of 15 minutes, compared to vessels flushed with and stored in heparinized saline solution (Perrault et al., J. Thorac. Cardiovasc. Surg. 161 (1): 96-106.e2, 2021).

It surprisingly has been found from an analysis of the one-year clinical outcomes from the European DuraGraft® Registry after endoscopic vein harvesting versus open vein harvesting in patients undergoing coronary artery bypass grafting performed using DuraGraft® vascular graft treatment composition that MACCE rates in isolated CABG patients that underwent endoscopic vein harvesting were significantly lower than that achieved in patients that underwent open vein harvesting when the graft vessels were treated with DuraGraft® vascular graft treatment composition as an endothelial damage inhibitor. Accordingly, the kits provided herein can include DuraGraft® vascular graft treatment composition Solutions A and B as an endothelial damage inhibitor composition, in combination with an endoscopic vessel harvesting device. Harvested vessels treated with DuraGraft® vascular graft treatment composition as an endothelial damage inhibitor exhibit reduced graft failure, which may be attributable to the protection of the endothelial layer of the vessel during the procedure. It is known that even short-time storage in physiological saline solution impairs endothelial vascular function of saphenous vein grafts. DuraGraft® vascular graft treatment composition as an endothelial damage inhibitor does not impair endothelial vascular function of saphenous vein or radial artery grafts. Further, it may be that DuraGraft® vascular graft treatment composition as an endothelial damage inhibitor also reduced oxidative damage, so that any oxidative and/or physical damage caused during the harvesting is minimized or reversed compared to when the vessel is soaked in saline, so that the implanted vessels exhibit the same or better patency as observed with vessels harvested using open harvesting methods.

Some medical indications require a product with a lower SAL, such as 10⁻⁶. A powder formulation, as described in U.S. Provisional Application 63/331,777, filed Apr. 15, 2022, describes a powder formulation that can be used as an endothelial damage inhibition composition that can be terminally sterilized allowing the SAL of the product to be reduced to 10⁻⁶, resulting in a product with decreased bioburden risk as well as generating a product that can be used in indications requiring a SAL of 10⁻⁶. The terminal sterilization can include gamma irradiation of the packaged powder formulation, but the sterilization process is not limited thereto and any suitable ionizing radiation sources can be used. The powder formulation can be easily packaged with an endoscopic vessel harvesting device to form certain embodiments of the kits provided herein.

In some exemplary embodiments, the powder formulation of the endothelial damage inhibition composition can be initially reconstituted into a small volume of sterile water followed by reconstitution in saline solution. The powder formulation of the endothelial damage inhibition composition can be initially reconstituted in about 5 ml to about 300 ml of sterile water, preferably about 10 ml to about 200 ml, about 15 ml to about 100 ml, about 20 ml to about 50 ml, and the like, followed by reconstitution in saline solution. The volume of the sterile water used in the initial reconstitution step is not limited hereto, and can include any values within any of the recited ranges and/or combinations thereof.

The components of the powder formulation of the endothelial damage inhibition composition can be included in their anhydrous forms, but are not limited thereto, and were developed for reconstitution at point of use using a saline solution, a salt solution and/or a water-for-injection (WFI) medium.

In some embodiments, the powder formulation of the endothelial damage inhibition composition includes reduced glutathione; a reducing agent such as ascorbic acid; a sugar such as D-glucose; a nitric oxide substrate such as L-arginine; a buffering agent, and a physiologically acceptable salt. In some embodiments, cysteinylglycine can be used as a replacement for the reduced glutathione, or a mixture of cysteinylglycine and reduced glutathione can be used in the powder formulation. Any suitable sugar can be used in place of glucose, but preferably any monosaccharide including, but not limited to, fructose, mannose and ribose can be used. In another exemplary embodiment, the powder formulation of the endothelial damage inhibition composition can include a reduced glutathione, a reducing agent, a buffering agent, and a physiologically acceptable salt.

Physiologically acceptable salts are those that are capable of forming a balanced salt solution when reconstituted in an aqueous medium. A typical balanced salt solution generally comprises water, calcium ions, chloride ions, potassium ions, phosphate ions, magnesium ions and sodium ions. The physiologically acceptable salts can be any one or more selected from among: calcium chloride dihydrate, potassium chloride, potassium phosphate monobasic, magnesium chloride hexahydrate, magnesium sulfate heptahydrate, sodium chloride, sodium bicarbonate, sodium phosphate dibasic heptahydrate, and combinations thereof. The physiologically acceptable salts are not limited, and can be included in their anhydrous forms or in their various hydrated forms. In a preferred exemplary embodiment, the physiologically acceptable salts are included in their anhydrous forms. In an alternate exemplary embodiment, the physiologically acceptable salts can be in their hydrated forms, including any degree of hydration, such as dihydrates, trihydrates, hexahydrates, heptahydrates, and the like. The salts in the solution are intended for buffering (to maintain pH) and to maintain isotonicity with respect to vascular conduits and to maintain ionic balance. When reconstituted at a point of use, the physiologically acceptable salts will form a balanced salt solution at a concentration that will result in an isotonic solution. In an exemplary embodiment, the physiologically acceptable salts are included in the powder formulation in amounts that, when reconstituted in a 1125 ml of saline solution or WFI medium, produce a balanced salt solution including about 0.14 grams/liter calcium chloride dihydrate, about 0.4 grams/liter potassium chloride, 0.06 grams/liter potassium phosphate monobasic, about 0.1 grams/liter magnesium chloride hexahydrate, about 0.1 grams/liter magnesium sulfate heptahydrate, about 8 grams/liter sodium chloride, about 0.36 grams/liter sodium bicarbonate, and about 0.03 grams/liter sodium phosphate dibasic heptahydrate. This is an exemplary reconstituted formulation, and other exemplary formulations can independently include other hydrated or anhydrous forms of the various components of the powder formulation, can exclude one or more components, and/or one or more components, in powder or liquid form, can be added to the powder formulation during reconstitution. In another exemplary embodiment, the physiologically acceptable salts are included in the powder formulation in amounts such that the pH of the reconstituted powder formulation at point of use can be about 7.0. In some embodiments, the pH can be about 7.3±0.4. The pH at the point of use can be adjusted using any known pH adjusting agents. Preferred pH adjusting agents are 4N HCl to decrease the pH and 84% NaHCO₃ to increase the pH.

The organic components are intended to maintain additional buffering capability, osmolality, to provide a non-oxidizing environment for tissues, organs and cells, and when arginine is present to maintain NO levels and prevent storage lesions in vascular grafts when outside of the body. The four organic components; L-glutathione, L-ascorbic acid, L-arginine and D-glucose, are normal constituents of blood and are also included for their roles in preserving and maintaining the extracellular environment of vascular conduits.

L-glutathione and L-ascorbic acid are antioxidants that prevent oxidative damage to cells by reacting with or neutralizing free radicals. The functions of these antioxidants in an endothelial damage inhibitor composition are: 1) to stabilize other components of the solution by preventing oxidation, thereby improving the stability and shelf life of product; and 2) to prevent oxidative damage to cell membranes, cellular components and extracellular matrix structures. Oxidative damage has been shown to damage the structural integrity of the extracellular architecture, thereby causing an interruption in the endothelial cell lining. Oxidative damage is also the basis for reperfusion injury that occurs following transplantation. Reperfusion injury exacerbates oxidative damage. The sugar in the formulation is intended to provide an energy source to support anaerobic metabolism for the tissue under ischemic/hypoxic conditions.

In an exemplary embodiment, the powder formulation of the endothelial damage inhibitor composition includes potassium chloride, potassium phosphate, magnesium chloride, magnesium sulfate, sodium bicarbonate, sodium phosphate, reduced L-glutathione, D-glucose, L-arginine and L-ascorbic acid. In another exemplary embodiment, the powder formulation includes potassium chloride, potassium phosphate monobasic, magnesium chloride hexahydrate, magnesium sulfate heptahydrate, sodium bicarbonate, sodium phosphate dibasic, reduced L glutathione, D-glucose, L-arginine and L-ascorbic acid. In another exemplary embodiment, the powder formulation includes potassium chloride, potassium phosphate, magnesium chloride, magnesium sulfate, sodium bicarbonate, sodium phosphate, reduced L-glutathione, D-glucose, L-arginine and L-ascorbic acid. In some embodiments, the powder formulation does not include sodium chloride (NaCl) prior to reconstitution, allowing saline to be used for reconstitution. In another exemplary embodiment, one or more of the components of the powder formulation may be added as a liquid during the manufacturing process and/or one or more of the components of the powder formulation may be provided as a liquid formulation that is added at the time of reconstitution. In yet another exemplary embodiment, calcium chloride is added as a separate liquid component that is added during reconstitution.

When reconstituted, the concentration of L-arginine should be from about 250 μM to about 2000 μM, glucose should be about 5 mM to about 50 mM, L-glutathione should be about 50 μM to about 3000 μM, and L-ascorbic acid should be about 500 μM to about 4000 μM. The concentration of L-arginine can be from about 300 μM to about 1750 μM, about 350 μM to about 1500 μM, about 400 μM to about 1250 μM, about 500 μM to about 1000 μM, and the like. The concentration of the glucose can be from about 5.1 mM to about 45 mM, about 5.2 mM to about 40 mM, about 5.3 mM to about 35 mM, about 5.4 mM to about 30 mM, about 5.5 mM to about 25 mM, about 5 mM to about 20 mM, and the like. The concentration of the L-glutathione can be from about 50 μM to about 2500 μM, about 60 μM to about 2000 μM, about 65 μM to about 1500 μM, about 70 μM to about 1000 μM, and the like. The concentration of the L-ascorbic acid can be about 550 μM to about 3500 μM, about 600 μM to about 3000 μM, about 650 μM to about 2500 μM, about 700 μM tot about 2000 μM, and the like. The amount(s) of the various components are not limited hereto, and can include any values within any of the recited ranges and/or combinations thereof.

The kits containing an endothelial damage inhibitor composition and an endoscopic vessel harvesting device are not limited to use with a particular tissue or organ. For example, the kits can be used for harvesting saphenous veins, internal thoracic/mammary arteries, epigastric arteries, gastroepiploic arteries and radial arteries, which then can be used, for example, in coronary bypass grafting (CABG) or peripheral bypass surgery. The vein or artery harvested using endoscopic harvesting can be used as a harvested bypass conduit.

Additionally, it is contemplated that the reconstituted solution of the endothelial damage inhibitor composition, whether prepared from the two-solution formulation, commercially available as DuraGraft® vascular graft treatment composition, or the powder composition described above, can be used to wash and bathe tissues and organs that have not been removed from the patient. For example, it is contemplated that the endothelial damage inhibitor composition can be used as a replacement for CO₂ insufflation, and can be used to create a subcutaneous tunnel to facilitate vessel harvesting. The use of the reconstituted endothelial damage inhibitor composition instead of CO₂ can minimize any localized pH damage that can occur with CO₂ insufflation, and also can prevent any oxidative damage.

An exemplary process for manufacturing a powder formulation of an endothelial damage inhibitor composition is described herein, but this process should not be considered to be a limiting disclosure, and the process can be subject to various parameter modifications as required to achieve the inventive aspect of the powder formulations. An exemplary process for manufacturing powder formulations according to an exemplary embodiment includes: a first step of screening components of the powder formulation including L-glutathione, L-glucose, L-arginine, L-ascorbic acid, calcium chloride anhydrous, potassium phosphate monobasic, magnesium sulfate anhydrous, magnesium chloride anhydrous, sodium phosphate dibasic anhydrous by passing through a mesh screen to yield components within a targeted particle size. As an example a #30 mesh screen can be used to remove particles larger than 595 μm, or a #35 mesh screen to remove particles larger than 500 μm, or a #40 mesh screen to remove particles larger than 400 μm. After the screening process is completed, the screened components are mixed or blended together. A second optional step of screening additional components of the powder formulation including potassium chloride and sodium bicarbonate by passing through a #30 mesh screen, #35 mesh screen, or #40 mesh screen can be completed. Typically, but not always, the same mesh size is used for all of the components to minimize particle segregation. In another exemplary embodiment, the first and second steps can be performed simultaneously. In another exemplary embodiment, each of the components, or any combination thereof, can be screened in separate steps prior to mixing. In an exemplary method, after mixing the components of the powder formulation together to form a mixture, the mixture was milled using a Fritz Mill (Screen #1722-0020) at 6000 rpm with knives forward to form a milled mixture. The milled mixture can be loaded into appropriate storage containers and sealed, such as filling into glass vials, closing with a rubber stopper and securing the rubber sopper with a flip-off/tear-off combination seal. The formulation also can be placed in a sealable pouch. This exemplary process can include an optional step of milling one or more of the components of the powder formulation prior to an initial step of screening the various components using a mesh screen, as described above.

The resulting containers of the endothelial damage inhibitor composition then can be sterilized. For example, terminal sterilization by gamma irradiation can be carried out to achieve a Sterility Assurance Level (SAL) of <10⁻⁶. For example, an ExCell® Irradiator manufactured by Sterigenics International Inc. (Oak Brook, IL) can be used. The number of vials and number of pouches can be selected to achieve a target density placed inside the ExCell® Irradiator and exposed to gamma radiation. Irradiation can be carried out at a range of 15 kGy to 25 kGy. It was determined that gamma radiation did not impact the stability of the powder formulations.

### Endoscopic Vessel Harvesting Device

The kits provided herein include as a component an endoscopic vessel harvesting device. Any known endoscopic vessel harvesting device can be included as a component of the kits.

Exemplary endoscopic methods and devices for performing vessel harvesting are discussed in detail, e.g., in U.S. Pat. Nos. 5,895,353, 6,162,173, 6,406,425, 6,471,638, 7,510,562, 7,662,162, 7,695,470, 8,480,696, and 11,141,055. Commercial vessel harvesting systems currently are available from manufacturers such as Terumo Cardiovascular (Ann Arbor, MI) Getinge AB (Goteborg, Sweden), and Saphena Medical (West Bridgewater, MA). Specific examples include the VirtuoSaph® and VirtuoSaph® Plus Endoscopic Vein Harvesting Systems (Terumo Cardiovascular, Ann Arbor, MI) Vaso View® 7xB System and Vasoview Hemopro 2 (Getinge A B, Goteborg, Sweden), VascuClear (LivaNova, London, UK), and Venapax (Saphena Medical, West Bridgewater, MA).

The device can be a closed tunnel system that includes a balloon to occlude the access site and can use carbon dioxide to insufflate the dissection tunnel. The device can be an open system that does not occlude the access site or use carbon dioxide to insufflate the dissection tunnel. The device can include a spoon-shaped dissector tip or a bullet-shaped dissector tip (van Diepen, Ann. Surg. 260 (2): 402-408, 2014). The devices typically are designed to fit within a harvesting cannula, which also contains the endoscope used for the procedure. The device typically creates a tunnel around the vessel using coaxial dissection. Insufflation with CO₂ can be used. The harvesting device typically cuts the vessels and seals using a combination of heat and pressure, although surgical clips, bipolar cautery, harmonic scalpel, or concentrated thermal energy also can be used.

The kits provided herein can include any endoscopic vessel harvesting device known in the art. For example, any endoscopic vessel harvesting device or system produced by Cardio Medical, Getinge, Karl Storz, LivaNova, Maquet Holdings, Med Europe S.r.l., Medical Instruments Spa, Saphena Medical, or Terumo Cardiovascular can be included.

The endoscopic vessel harvesting device can include a vessel dissector for separating a blood vessel from surrounding tissue. The vessel dissector can have an elongated cannula having a blunt tip for separating layers of tissue from around the vessel. The tip of the vessel dissector can be movable, and is typically transparent to permit viewing forward of the tip using an endoscope. The endoscopic vessel harvesting device can include a vessel harvester. The vessel harvester can include a cannula that includes a lumen for receiving the endoscope. The vessel harvester can include one or more tools that are within the vessel harvester and that permit manipulation, cutting, and sealing of vessels. The vessel harvester can include, e.g., a vessel holding tool and a vessel cutting tool. The vessel holding tool can allow vessel position manipulation. The vessel holding tool can include a vessel hook that can be used to capture a target vessel. The endoscopic vessel harvesting device can include a trocar. Many endoscopic harvesting devices currently in use include a vessel dissector, a vessel harvester, and a trocar. An endoscope can extend through the entire length of the vessel dissector to allow a viewing end to terminate in proximity to the tip of the vessel dissector, which typically is conical and transparent. Most vessel harvesting systems are disposable and are packaged and sold separately from the endoscope which is typically reused. In all cases, however, these endoscopic vessel harvesting systems are designed to accommodate and function with a conventional endoscope.

The vessel harvester includes cutting tools to cut side branches from the target vessel. Typically the cutting tool can be configured to simultaneously seal the severed side branches to prevent bleeding. In some configurations, a holding tool can be included in the vessel harvester to manipulate the position of the target vessel so that the cutting tool can access the side branches. Control of these tools usually is located in a handle of the endoscopic vessel harvesting device. In some configurations, an insufflation system that includes tubing to provide a gas such as CO₂ is included so that the gas can exit an open distal end of the cannula to facilitate vessel harvesting by separating the vessel from surrounding tissue. Monopolar and/or bipolar probes, forceps or scissors that use high frequency electrical current can be used to allow the electrical current to pass through the tissue to seal the tissue and prevent bleeding by causing the tissue to be heated and the tissue proteins to coagulate.

### Methods

Also provided are methods of improving the quality of an endoscopically harvested vessel. The methods include reconstituting the endothelial damage inhibitor composition of the kits disclosed herein in saline or a sterile water media to form a reconstituted solution, and soaking the endoscopically harvested vessel in the reconstituted solution for at least 15 minutes. The endoscopically harvested vessel can be soaked for any necessary period of time between extraction of the vessel and use in surgery. The period of time can be from 15 minutes to 240 minutes, or 20 to 180 minutes, or 25 to 120 minutes, or 30 to 90 minutes The time can be 15 minutes, 20 minutes, 25 minutes, 30 minutes, 35 minutes, 40 minutes, 45 minutes, 50 minutes, 55 minutes, or 60 minutes. The methods can further include flushing the endoscopically harvested vessel with the reconstituted solution after harvesting and prior to soaking. For the flushing, an aliquot of the reconstituted solution can be set aside for flushing, and the remainder used for soaking the endoscopically harvested vessel. The methods can further include flushing the endoscopically harvested vessel with the reconstituted solution after soaking. For the flushing after soaking, an aliquot of the reconstituted solution can be set aside for the flushing before the remainder of the reconstituted solution is used for soaking the endoscopically harvested vessel. As discussed below in the examples, it has surprisingly been found that by flushing and storing the harvested vessel in an endothelial damage inhibitor described herein, at one year, the MACCE rates in patients undergoing endoscopic vessel harvesting were significantly lower when compared to patients receiving vessels harvested using open vessel harvesting.

Also provided are methods of reducing damage of an endoscopically harvested vessel, the methods including reconstituting the endothelial damage inhibitor composition of the kits provided herein in saline or a sterile water media to form a reconstituted solution, and administering the reconstituted solution via the endoscopic vessel harvesting device of the kits as a replacement for CO₂ insufflation or in conjunction with CO₂ insufflation to create a subcutaneous tunnel to facilitate vessel harvesting. The use of the reconstituted endothelial damage inhibitor composition instead of CO₂ or in conjunction with can minimize any localized pH damage that can occur with CO₂ insufflation, and also can prevent any oxidative damage.

### Examples

### 1. Analysis of One-Year Clinical Outcomes

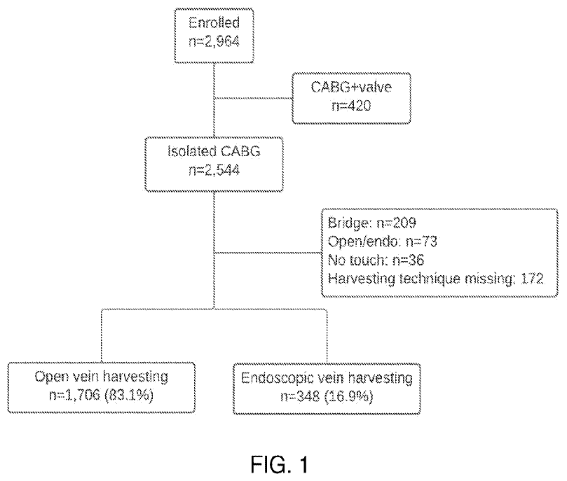

An analysis of the one-year clinical outcomes after endoscopic vein harvesting versus open vein harvesting in patients undergoing coronary artery bypass grafting was performed using the results from the European DuraGraft® Registry. The patients were from a large prospective multi-center all-comer European CABG registry (n=2,964). There were 45 sites in 8 countries (Austria, Germany, Ireland, Italy, UK, Spain, Switzerland, and Turkey). The subgroup analysis was of isolated CABG patients (n=2,544) grouped into Open Vein Harvesting (OVH: n=1,706) and Endoscopic Vein Harvesting (EVH: n=348), where an SVG or free arterial graft was flushed and stored intra-operatively in DuraGraft® vascular graft treatment composition, an endothelial damage inhibitor. The chart of the patient groupings is shown in FIG. 1.

The objective of this study was to compare open vein harvesting versus endoscopic vein harvesting in patients undergoing isolated CABG and whose saphenous vein grafts had been treated with an endothelial damage inhibitor. A 1:1 propensity score matching (PSM) was used to balance for differences in baseline and procedural characteristics. Event rates were based on Kaplan-Meier estimates.

The patient and surgical characteristics of the PSM matched population is shown in Table 1 below.

TABLE 1

| | Unweighted | | | Weighted | | |
|---|---|---|---|---|---|---|
| | Open | Endoscopic | | Open | Endoscopic | |
| | n = 1,706 | n = 348 | P-value | n = 333 | n = 333 | P-value |
| Age | 67.0 ± 9.0 | 67.5 ± 9.8 | 0.40 | 67.9 ± 8.7 | 67.4 ± 9.9 | 0.48 |
| Caucasian | 87.3 | 81.0 | <0.01 | 79.9 | 80.5 | 0.92 |
| Male | 82.7 | 81.3 | 0.54 | 83.2 | 80.8 | 0.48 |
| LVEF¹ < 30% | 5.1 | 4.6 | 0.70 | 6.6 | 4.8 | 0.40 |
| EuroScore II² | 2.3 ± 3.7 | 2.1 ± 2.3 | 0.13 | 2.3 ± 4.0 | 2.1 ± 2.4 | 0.55 |
| Chronic renal | 3.8 | 2.0 | 0.11 | 2.1 | 2.1 | 1.00 |
| failure | | | | | | |
| Insulin dependent | 15.6 | 11.2 | 0.11 | 11.1 | 11.7 | 0.76 |
| diabetes mellitus | | | | | | |
| Urgent/emergent | 26.1 | 17.5 | <0.001 | 21.9 | 18.3 | 0.29 |
| surgery | | | | | | |
| On-pump | 79.7 | 92.8 | <0.001 | 92.5 | 92.5 | 1.00 |
| # of arterial grafts/ | 1.2 ± 0.5 | 1.0 ± 0.6 | <0.001 | 1.1 ± 0.6 | 1.1 ± 0.6 | 0.95 |
| patient | | | | | | |
| # of vein grafts/ | 1.6 ± 0.8 | 1.7 ± 0.8 | 0.42 | 1.8 ± 0.9 | 1.7 ± 0.9 | 0.18 |
| patient | | | | | | |
| LIMA graft | 94.7 | 82.2 | <0.001 | 85.3 | 85.9 | 0.91 |

¹Left ventricular ejection fraction, which is the measure of the left ventricular systolic function.
²European System for Cardiac Operative Risk Evaluation, which can be used to predict the risk of in-hospital mortality after major cardiac surgery.

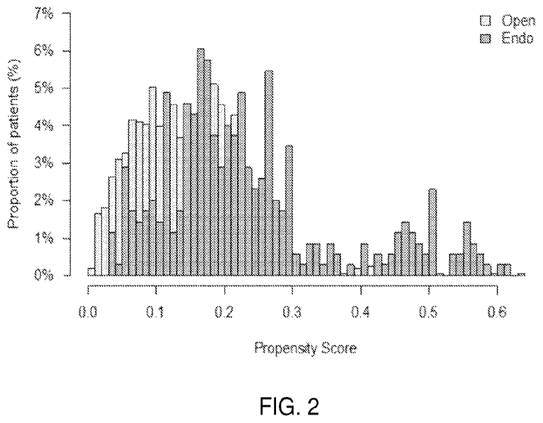

As can be seen in the table, after PMS matching, a good balance in baseline and procedural characteristics between the groups was achieved. This also is shown in FIG. 2, which is a histogram of propensity scores demonstrating good matching between groups.

A survival analysis was performed using the Kaplan-Meier (KM) estimate to measure the fraction of subjects living for a certain time after treatment. A guide to understanding Kaplan-Meier curves is available (Rich et al., Otolaryngol. Head Neck Surg. 143 (3): 331-336, 2010). The KM estimated outcome rates at 1-year after surgery (after propensity score matching) are shown in Table 2 below.

TABLE 2

| | Open | Endoscopic | | |
|---|---|---|---|---|
| | n = 333 | n = 333 | HR (95% CI) | p-value |
| MACE¹ | 7.6 | 4.0 | 1.9 (1.0-3.8) | 0.05 |
| MACCE² | 8.8 | 4.6 | 2.0 (1.1-3.7) | 0.03 |
| All death | 6.4 | 3.4 | 1.9 (0.9-4.0) | 0.09 |
| MI | 1.3 | 1.0 | 1.4 (0.3-6.2) | 0.67 |
| Revascularization | 1.6 | 0.6 | 2.6 (0.5-13.4) | 0.25 |
| Stroke | 2.0 | 1.6 | 1.3 (0.4-4.1) | 0.71 |

¹= Major Adverse Cardiovascular Events (which is a composite of all death, myocardial infarction, and revascularization (requiring another surgery to replace the vessel))
²= Major Adverse Cardiovascular and Cerebrovascular Events (MACE plus stroke)

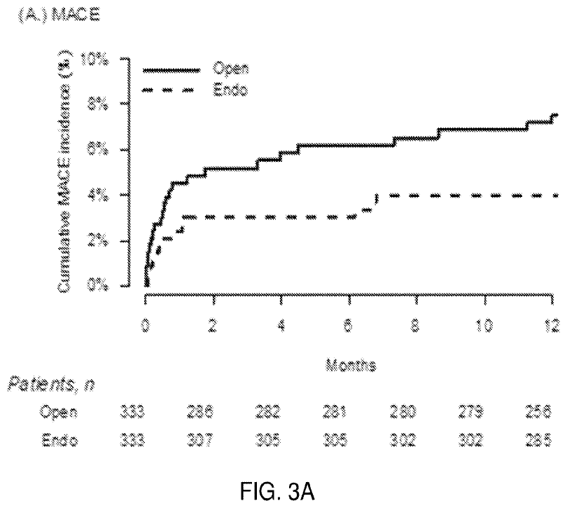

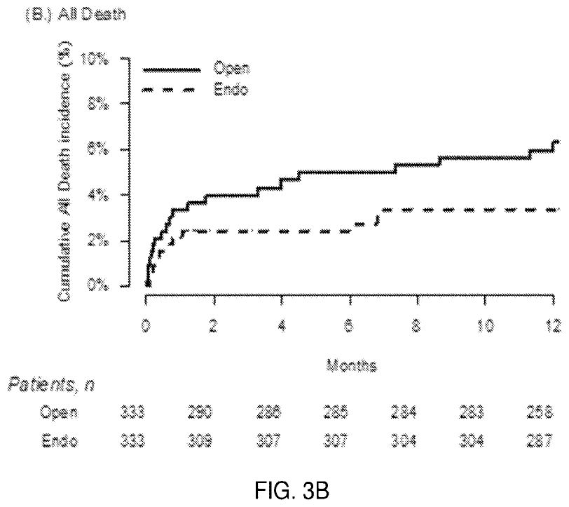

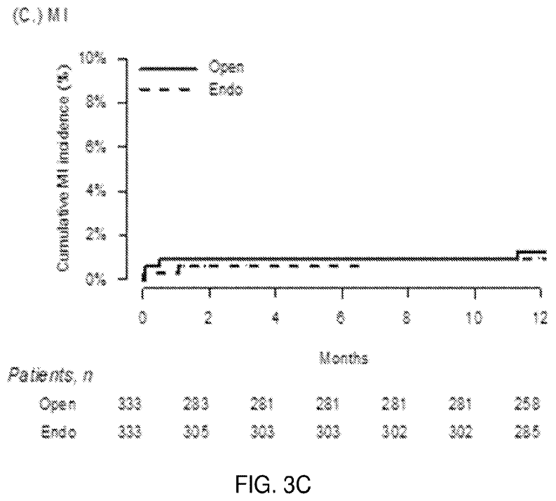

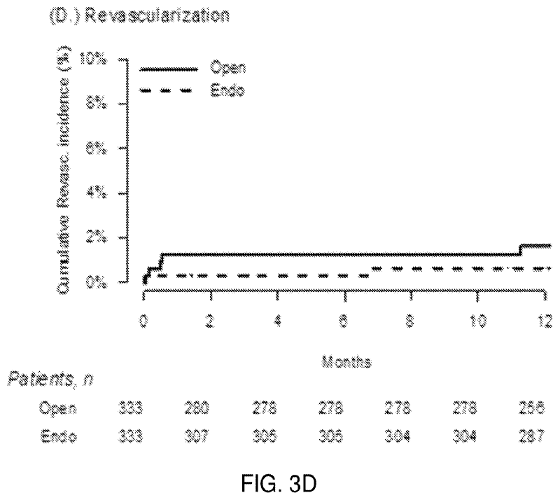

MACE and MACCE are useful endpoints to evaluate cardiovascular outcomes after coronary artery bypass grafting (CABG). The graphs for MACE, all deaths, MI, and revascularization are shown in FIG. 3A, FIG. 3B. FIG. 3C and FIG. 3D. These results demonstrate that when a harvested saphenous vein was treated with an endothelial damage inhibitor, such as DuraGraft® vascular graft treatment composition, patients that received veins harvested using endoscopic vein harvesting exhibited better primary (MACE) and secondary (MACCE) outcome measures. In particular, as can be seen from the data, at 1 year, MACCE rates in isolated CABG patients that underwent endoscopic vein harvesting were significantly lower than that achieved in patients that underwent open vein harvesting when the graft vessels were treated with DuraGraft® vascular graft treatment composition as an endothelial damage inhibitor.

The results are surprising because open vein harvesting typically results in graft vessels that exhibit better patency than vessels harvested using endoscopic harvesting. In this multi European registry, endoscopic vein harvesting (EVH) was associated with a significantly lower 1-year MACCE rate compared to open vein harvesting (OVH). Additional follow-up (up to 5 years) is underway to assess whether EVH is associated with better long-term outcomes in this registry.

2. Analysis of vascular parameters in vein grafts prepared by EVH and Open Harvest

A prospective study was performed which showed that pretreatment of saphenous vein grafts (SVG) with DuraGraft® vascular graft treatment composition, an endothelial damage inhibitor, mitigated negative remodeling by improvement of several vascular parameters, when compared to pretreatment with saline alone. The protocol for the study was the same as that previously published (Ali, et al., Open Heart 2018; 5: e000780. doi: 10.1136/openhrt-2018-000780).

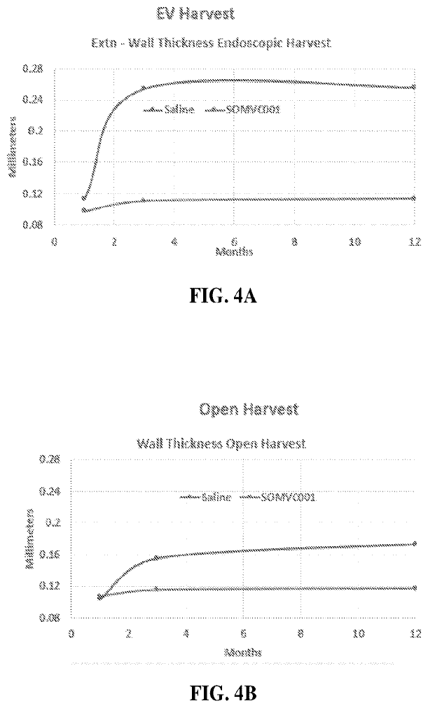

The study showed that pretreatment with DuraGraft® vascular graft treatment composition (referred to as SOMVC001) resulted in a decrease in wall thickness using either EVH (FIG. 4A) or open (FIG. 4B) harvesting methods compared to using saline alone.

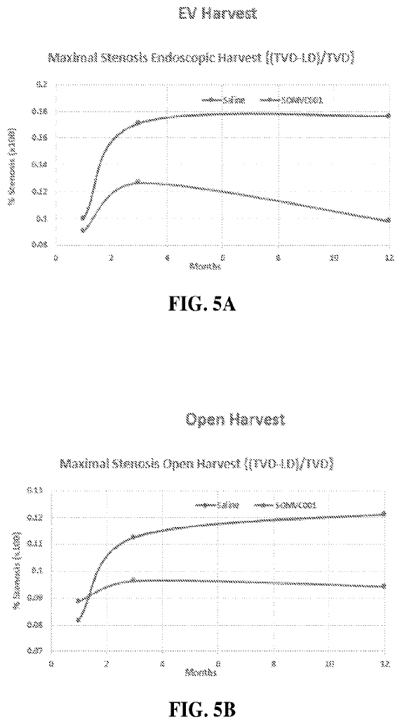

In addition, the study showed that pretreatment with DuraGraft® vascular graft treatment composition (referred to as SOMVC001) resulted in a decrease in maximal stenosis using either EVH (FIG. 5A) or open (FIG. 5B) harvesting methods compared to using saline alone.

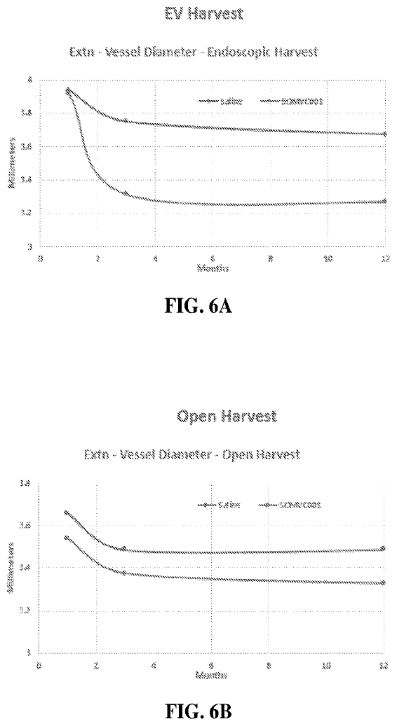

Finally, the study showed that pretreatment with DuraGraft® vascular graft treatment composition (referred to as SOMVC001) resulted in a decrease in vessel diameter using either EVH (FIG. 6A) or open (FIG. 6B) harvesting methods compared to using saline alone.

As various changes could be made in the above formulations and methods without departing from the scope of the invention, it is intended that all matter contained in the above description shall be interpreted as illustrative and not in a limiting sense.

1. A kit, comprising:
a) an endothelial damage inhibitor composition; and
b) an endoscopic vessel harvesting device.

2-28. (canceled)
